(19) United States
(12) Patent Application Publication
(10) Pub. No.: US 2025/0319285 A1
(43) Pub. Date: Oct. 16, 2025

(54) NEEDLE TUBE STRUCTURE AND ENDOSCOPIC INTERVENTIONAL INSTRUMENT

(71) Applicant: Micro-Tech (Nanjing) Co., Ltd., Nanjing (CN)

(72) Inventors: Chengyou Ye, Nanjing (CN); Zhi Tang, Nanjing (CN); Lan Zhang, Nanjing (CN); Hailang Gu, Nanjing (CN); Yongan Zou, Nanjing (CN)

(21) Appl. No.: 19/172,903

(22) Filed: Apr. 8, 2025

(30) Foreign Application Priority Data
Apr. 12, 2024 (CN) 2024207640953

(51) Int. Cl. A61M 25/09; A61M 25/06

(52) U.S. Cl. CPC A61M 25/09041; A61M 25/065

(57) ABSTRACT

A needle tube structure and an endoscopic interventional instrument are disclosed that relate to the technical field of endoscopic medical instruments. The needle tube structure provided in the present disclosure includes a needle tube body and a polymer material tube body provided inside the needle tube body. The side wall of the needle tube body is provided with at least one slot, and the polymer material tube body is fixedly fitted to the slot. The needle tube structure and the endoscopic interventional instrument can ensure that the polymer material tube body is firmly joined to the needle tube body, which not only prevents a guide wire passing through the needle tube body from being scratched or damaged by the needle tube body but also enhances the stability of the polymer material tube body, thus reducing the risk of detachment of the polymer material tube body.

### CROSS-REFERENCE TO RELATED APPLICATIONS

The present disclosure claims the priority to the Chinese patent application with the filling No. 2024207640953, filed on Apr. 12, 2024, the entire content of which is incorporated herein by reference for all purposes.

### TECHNICAL FIELD

The present disclosure relates to the technical field of endoscopic medical instruments, and particularly to a needle tube structure and an endoscopic interventional instrument.

### BACKGROUND ART

The endoscopic interventional instrument arranges a passage for the insertion of a guide wire. The guide wire needs to reciprocate along the needle tube of the endoscopic interventional instrument to arrange the distal end of the guide wire at the target location. Since the front end of the needle tube needs to be provided with a needle tip blade configured for cutting or puncturing, when the needle tube slides along the guide wire, the needle tip blade will cut or scrape the guide wire, easily stripping the surface layer of the guide wire and even causing severe damage to the guide wire. In addition, a cushion layer is inserted in the inner side of the needle tip blade to separate the guide wire from the needle tube, thereby preventing the needle tip blade of the needle tube from scraping the surface layer of the guide wire. However, in medical applications, the cushion layer on the inner side of the needle tip blade has a high risk of detachment, which may lead to medical accidents caused by the cushion layer being left inside the human body.

### SUMMARY

The objective of the present disclosure is to provide a needle tube structure and an endoscopic interventional instrument to address the technical problems of the needle tube structure easily scraping the surface layer of the guide wire and the polymer material tube body being prone to detachment.

In a first aspect, the present disclosure provides a needle tube structure, comprising a needle tube body and a polymer material tube body provided inside the needle tube body, wherein a side wall of the needle tube body is provided with at least one slot, and the polymer material tube body is fixedly fitted to the slot.

In connection with the first aspect, the present disclosure provides a first possible embodiment of the first aspect, where the polymer material tube body is inserted inside the needle tube body, and the polymer material tube body is fixed to the needle tube body by an adhesive material injected from the slot.

In connection with the first aspect, the present disclosure provides a second possible embodiment of the first aspect, where the needle tube body and the polymer material tube body are formed by a co-injection molding process to join the needle tube body and the polymer material tube body.

In connection with the first aspect, the present disclosure provides a third possible embodiment of the first aspect, where at inner side wall of the needle tube body, the polymer material tube body is formed by spraying to allow the polymer material tube body to be joined and attached to the inner side wall of the needle tube body.

In connection with the first aspect, the present disclosure provides a fourth possible embodiment of the first aspect, the needle tube body and the polymer material tube body are integrally molded by a hot-melt process.

In connection with the first aspect, the present disclosure provides a fifth possible embodiment of the first aspect, the polymer material tube body forms a protruding portion fitted to the slot.

In connection with the first aspect, the present disclosure provides a sixth possible embodiment of the first aspect, an end of the needle tube body is provided with a needle tip blade, and the polymer material tube body is provided with an incision portion covering an inner side of the needle tip blade.

In connection with the first aspect, the present disclosure provides a seventh possible embodiment of the first aspect, the needle tip blade and the incision portion are flush along a blade surface, and the blade surface is arranged obliquely relative to an axis of the needle tube body; and an inner edge of the incision portion near the axis of the needle tube body is exposed from the blade surface.

In a second aspect, an endoscopic interventional instrument is provided in the present disclosure, including: a guide wire and the needle tube structure as described in the first aspect, wherein the guide wire passes through the needle tube structure, allowing the guide wire to be able to slide along the needle tube structure.

In connection with the second aspect, the needle tube body is inserted into an outer tube in a sliding manner.

The embodiment of the present disclosure offers beneficial effects as follows. A polymer material tube body is arranged inside the needle tube body, and a side wall of the needle tube body is provided with at least one slot, and the polymer material tube body is fixedly fitted to the slot. This can ensure that the polymer material tube body is firmly joined to the needle tube body, which not only prevents a guide wire passing through the needle tube body from being scratched or damaged by the needle tube body but also enhances the stability of the polymer material tube body, thus reducing the risk of detachment of the polymer material tube body.

In order to make the above objective, features, and advantages of the present disclosure more obvious and easier to understand, the following is a better example, and with the attached drawings, for detailed description as follows.

### BRIEF DESCRIPTION OF DRAWINGS

To more clearly illustrate the technical solutions in the specific embodiments of the present disclosure or related technologies, a brief introduction to the drawings required in the description of the specific embodiments or related technologies is provided below. Obviously, the drawings described below are some embodiments of the present disclosure, for those of ordinary skill in the art, without paying inventive effort, may also obtain other drawings according to these drawings.

FIG. 1 is a partial schematic diagram of a needle tube structure and an outer tube of an endoscopic interventional instrument provided in the embodiment of the present disclosure;

FIG. 2 is an enlarged schematic diagram of position A in FIG. 1;

FIG. 3 is a partial schematic diagram of another needle tube structure and an outer tube of an endoscopic interventional instrument provided in the embodiment of the present disclosure;

FIG. 4 is an enlarged schematic diagram of position B in FIG. 3; and

FIG. 5 is a partially enlarged schematic diagram of a needle tube body of an endoscopic interventional instrument provided in the embodiment of the present disclosure.

Reference numerals: 100—needle tube body; 101—slot; 102—needle tip blade; 200—polymer material tube body; 201—protruding portion; 202—incision portion; 203—inner edge; 300—outer tube.

### DETAILED DESCRIPTION OF EMBODIMENTS

A clear and complete description of the technical solutions of the present disclosure will be given below in connection with the drawings. Obviously, the described embodiments are a portion of the embodiments of the present disclosure and not all of the embodiments. Based on the embodiments in the present disclosure, all other embodiments obtained by those of ordinary skill in the art without making inventive efforts are within the scope of protection of the present disclosure.

In the description of the present disclosure, it should be noted that the terms “center”, “upper”, “lower”, “left”, “right”, “vertical”, “horizontal”, “inner”, “outer”, and other positional or directional indications are based on the orientations or positional relationships shown in the drawings. They are only provided for ease of description and simplification of the present disclosure and should not be understood as indicating or implying that the referred devices or components must have a specific orientation, be configured, or operate in a specific orientation. Therefore, they should not be construed as limitations on the present disclosure. In addition, the terms “first”, “second”, and “third” are only used to distinguish names and should not be understood as indicating or implying relative importance. Unless otherwise specified, physical quantities in formulas should be understood as fundamental quantities in the International System of Units (SI) or as derived quantities obtained through mathematical operations such as multiplication, division, differentiation, or integration based on fundamental quantities.

In the description of the present disclosure, it is important to note that unless otherwise clearly stipulated and limited, the terms “mount”, “interconnect” and “connect” should be understood in a broad sense, for example, it can be a fixed connection, a detachable connection, or an integral connection; it can be a mechanical connection or an electrical connection; and it can be a direct connection, an indirect connection through an intermediary, or an internal communication between two components. Those of ordinary skill in the art can understand the meanings of the above terms in the present disclosure according to specific situations.

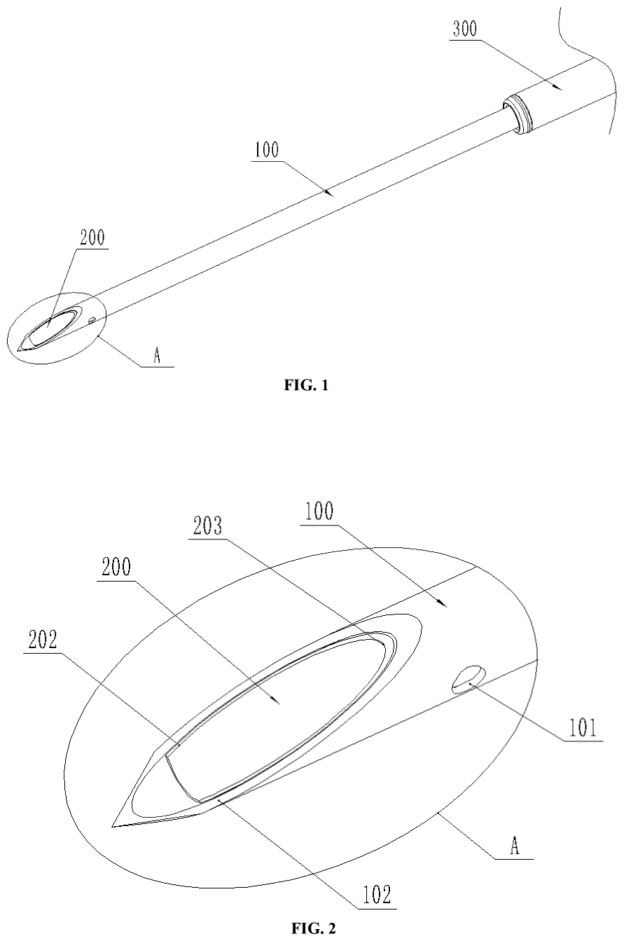

As shown in FIGS. 1, 3, and 5, the needle tube structure provided in the present disclosure includes a needle tube body 100 and a polymer material tube body 200 provided inside the needle tube body 100. The side wall of the needle tube body 100 is provided with at least one slot 101, and the polymer material tube body 200 is fixedly fitted to the slot 101.

In the embodiment, the polymer material tube body 200 is made of materials such as fiber, resin, plastic, and rubber that are harmless to the human body. When the needle tube structure is in use, the guide wire passes through the needle tube body 100 along the axial direction, and the polymer material tube body 200 is arranged between the guide wire and the needle tube body 100 to prevent the needle tube body 100 from scraping the surface layer of the guide wire. Furthermore, the side wall of the needle tube body 100 is provided with at least one slot 101, and the polymer material tube body 200 can be fitted to the slot 101. Reinforcement material can be added through the slot 101 to improve the joint strength between the polymer material tube body 200 and the needle tube body 100, thereby enhancing the stability of the polymer material tube body 200 and reducing the risk of detachment of the polymer material tube body 200, which may lead to medical accidents.

As shown in FIG. 1 and FIG. 2, in a first optional embodiment, the polymer material tube body 200 is inserted inside the needle tube body 100, and the polymer material tube body 200 is fixed to the needle tube body 100 by an adhesive material injected from the slot 101. The adhesive solution can penetrate the gap between the outer wall of the polymer material tube body 200 and the inner wall of the needle tube body 100 through the slot 101, thereby ensuring a stable bonding between the polymer material tube body 200 and the needle tube body 100.

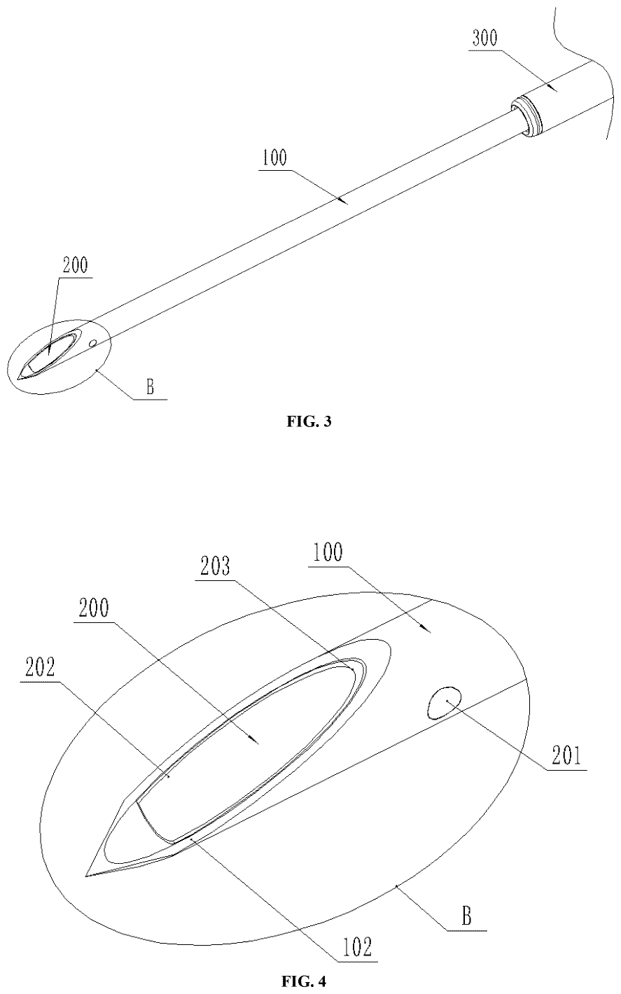

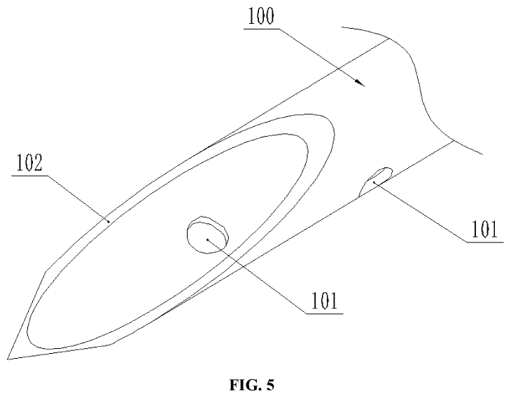

As shown in FIG. 3 and FIG. 4, in a second optional embodiment, the needle tube body 100 and the polymer material tube body 200 are integrally molded through a co-injection molding process, so that the needle tube body 100 and the polymer material tube body 200 are joined. This enables simultaneous molding of the needle tube body 100 and the polymer material tube body 200 while ensuring a secure joint between them, thereby improving processing efficiency.

In a third optional embodiment, at inner side wall of the needle tube body 100, the polymer material tube body 200 is formed by spraying to allow the polymer material tube body 200 to be joined and attached to the inner side wall of the needle tube body 100. This method also allows for rapid processing and molding.

In a fourth optional embodiment, the needle tube body 100 and the polymer material tube body 200 are integrally molded through a hot-melt process, which further improves production efficiency and ensures a more stable joint between the needle tube body 100 and the polymer material tube body 200 after solidification.

Referring to FIG. 2 and FIG. 5, based on the above optional embodiments, the polymer material tube body 200 can form a protruding portion 201 that fits into the slot 101. By embedding the protruding portion 201 into the slot 101, the stability of the polymer material tube body 200 relative to the needle tube body 100 is enhanced, thereby reducing the risk of detachment of the polymer material tube body 200.

As shown in FIG. 2 and FIG. 4, the end of the needle tube body 100 is provided with a needle tip blade 102, wherein the needle tip blade 102 can cut and pierce the target human tissue. The polymer material tube body 200 includes an incision portion 202 covering the inner side of the needle tip blade 102. The incision portion 202 can separate the guide wire passing through the needle tube body 100 from the needle tip blade 102, thereby preventing damage to the surface layer of the guide wire.

Further, the needle tip blade 102 and the incision portion 202 are flush along a blade surface, and the blade surface is arranged obliquely relative to an axis of the needle tube body 100; and an inner edge 203 of the incision portion 202 near the axis of the needle tube body 100 is exposed from the blade surface. The blade surface includes a plane or an arc surface that is inclined relative to the axis of the needle tube body 100, and the inner edge 203 is exposed from the blade surface. In other words, the inner edge 203 can be in contact with the guide wire, thereby preventing the guide wire from contacting the inner edge of the needle tip blade 102. This prevents a guide wire that is bent relative to the axis of the needle tube body 100 from being scraped by the needle tip blade 102.

As shown in FIG. 1, an endoscopic interventional instrument is provided in the present disclosure, including: a guide wire and the needle tube structure as described in the embodiments, wherein the guide wire passes through the needle tube structure, allowing the guide wire to be able to slide along the needle tube structure. The needle tube body 100 can be implanted toward the distal end away from the operating end, thereby achieving a puncture of the target human tissue. Subsequently, the guide wire is inserted along the needle tube body 100. During this process, the polymer material tube body 200 is arranged between the guide wire and the needle tube body 100, which not only prevents wear between the guide wire and the needle tube body 100 but also avoids scraping damage to the surface layer of the guide wire caused by the needle tube body 100. Additionally, the polymer material tube body 200 is firmly joined to the needle tube body 100, thus reducing the risk of detachment of the polymer material tube body 200 which may lead to medical accidents.

In the embodiment of the present disclosure, the needle tube body 100 is inserted into an outer tube 300 in a sliding manner, and the outer tube 300 is inserted into an endoscope. The endoscope enables the visual operation of the needle tube body 100. The needle tube body 100 moves telescopically relative to the outer tube 300. The outer tube 300 is used to preliminarily implant the needle tube body 100 to a specific location, after which the needle tube body 100 extends outward relative to the outer tube 300 to achieve puncture of the target human tissue. Then, the guide wire is inserted along the needle tube body 100, during which the polymer material tube body 200 prevents the needle tube body 100 from scraping the guide wire.

Finally, it should be noted that the above embodiments are only used to illustrate the technical solution of the present disclosure, not to limit it, and the present disclosure has been described in detail with reference to the preceding embodiments. It will be understood by those of ordinary skill in the art that, one may still modify the technical solution described in the preceding embodiments or replace some or all the technical features equally; these modifications or substitutions do not depart the essence of the corresponding technical solution from the scope of the technical solution of the embodiments of the present disclosure.

1. A needle tube structure, comprising:
a needle tube body and a polymer material tube body provided inside the needle tube body, wherein a side wall of the needle tube body is provided with at least one slot, and the polymer material tube body is fixedly fitted to the slot.

2. The needle tube structure according to claim 1, wherein the polymer material tube body is inserted inside the needle tube body, and the polymer material tube body is fixed to the needle tube body by an adhesive material injected from the slot.

3. The needle tube structure according to claim 1, wherein the needle tube body and the polymer material tube body are formed by a co-injection molding process to join the needle tube body and the polymer material tube body.

4. The needle tube structure according to claim 1, wherein at an inner side wall of the needle tube body, the polymer material tube body is formed by spraying to allow the polymer material tube body to be joined and attached to the inner side wall of the needle tube body.

5. The needle tube structure according to claim 1, wherein the needle tube body and the polymer material tube body are integrally molded by a hot-melt process.

6. The needle tube structure according to claim 1, wherein the polymer material tube body forms a protruding portion fitted to the slot.

7. The needle tube structure according to claim 1, wherein an end of the needle tube body is provided with a needle tip blade, and the polymer material tube body is provided with an incision portion covering an inner side of the needle tip blade.

8. The needle tube structure according to claim 7, wherein the needle tip blade and the incision portion are flush along a blade surface, and the blade surface is arranged obliquely relative to an axis of the needle tube body; and
wherein an inner edge of the incision portion near the axis of the needle tube body is exposed from the blade surface.

9. An endoscopic interventional instrument, comprising: a guide wire and the needle tube structure according to claim 1, wherein the guide wire passes through the needle tube structure, allowing the guide wire to be able to slide along the needle tube structure.

10. The endoscopic interventional instrument according to claim 9, wherein the needle tube body is inserted into an outer tube in a sliding manner.

11. The endoscopic interventional instrument according to claim 9, wherein the polymer material tube body is inserted inside the needle tube body, and the polymer material tube body is fixed to the needle tube body by an adhesive material injected from the slot.

12. The endoscopic interventional instrument according to claim 9, wherein the needle tube body and the polymer material tube body are formed by a co-injection molding process to join the needle tube body and the polymer material tube body.

13. The endoscopic interventional instrument according to claim 9, wherein at an inner side wall of the needle tube body, the polymer material tube body is formed by spraying to allow the polymer material tube body to be joined and attached to the inner side wall of the needle tube body.

14. The endoscopic interventional instrument according to claim 9, wherein the needle tube body and the polymer material tube body are integrally molded by a hot-melt process.

15. The endoscopic interventional instrument according to claim 9, wherein the polymer material tube body forms a protruding portion fitted to the slot.

16. The endoscopic interventional instrument according to claim 9, wherein an end of the needle tube body is provided with a needle tip blade, and the polymer material tube body is provided with an incision portion covering an inner side of the needle tip blade.

17. The endoscopic interventional instrument according to claim 16, wherein the needle tip blade and the incision portion are flush along a blade surface, and the blade surface is arranged obliquely relative to an axis of the needle tube body; and
an inner edge of the incision portion near the axis of the needle tube body is exposed from the blade surface.

18. The needle tube structure according to claim 2, wherein the polymer material tube body forms a protruding portion fitted to the slot.

19. The needle tube structure according to claim 3, wherein the polymer material tube body forms a protruding portion fitted to the slot.

20. The needle tube structure according to claim 4, wherein the polymer material tube body forms a protruding portion fitted to the slot.
